(19) United States
(12) Patent Application Publication
(10) Pub. No.: US 2004/0262419 A1
(43) Pub. Date: Dec. 30, 2004

(54) Breakable wick for use in a dispenser for a volatile liquid

(76) Inventors: Kara L. Kotary, Racine, WI (US); Ralph Schwarz, Racine, WI (US); Tyler D. Duston, Evanston, IL (US)

Correspondence Address: S.C. JOHNSON & SON, INC., 1525 HOWE STREET, RACINE, WI, 53403-2236, US

(21) Appl. No.: 10/607,702

(22) Filed: Jun. 27, 2003

(51) Int. Cl. A61L 9/04

(52) U.S. Cl. 239/44; 239/45; 239/51.5

(57) ABSTRACT

A wick assembly for use in a dispenser for a volatile liquid which includes a wick having an external surface and a discontinuity in the surface providing a location in the wick of less resistance to a force applied to the wick than a location adjacent to the discontinuity.

### FIELD OF THE INVENTION

The present invention relates to wicks for dispensing a volatile liquid and, more particularly, wicks which project from a container holding the volatile liquid.

### BRIEF DESCRIPTION OF THE DRAWINGS

FIG. 1 is a perspective view showing insertion of the wick of the present invention into a housing of a dispensing device;

FIG. 2 is a front schematic view of the dispenser assembled of FIG. 1 showing the dispenser housing partially cut away;

FIG. 2A is a side elevational view showing the dispenser assembled of FIG. 1 with the housing partially cut away;

FIG. 3 is a cross-sectional view taken along line c-c of FIG. 2A of the present invention;

FIG. 4 is a side elevational view of the breakable wick of the present invention;

FIG. 5 is a cross-sectional view of an embodiment of the wick taken along line 5-5 in FIG. 4;

FIG. 5a is an enlarged partial view of the wick shown in FIG. 5;

FIG. 6 is a cross-sectional view of a second embodiment of the wick shown in FIG. 4;

FIG. 6a is an enlarged partial view cross-sectional view of a central portion of the wick shown in FIG. 6;

FIG. 7 is showing breakage of the wick in FIG. 5 under application of a force to the wick;

FIG. 8 is a side elevational view of a third embodiment of the wick;

FIG. 9 is a cross-sectional view of an embodiment of the wick assembly showing the wick of FIG. 8 in a container for holding a volatile liquid to which it is secured;

FIG. 10 is a cross-sectional view of the third embodiment of the wick of FIG. 9 in which the wick is fitted inside a retention member;

FIG. 11 is a side elevational view of a fourth embodiment of the wick of the present invention;

FIG. 12 is a side elevational view of a fifth embodiment of the wick of the present invention;

FIG. 13 is a side view of a sixth embodiment of the wick of the present invention;

FIG. 14 is a cross-sectional view of the wick of FIG. 13 secured in a retention member mounted in a container for holding a volatile liquid;

FIG. 15 is a cross-sectional view of a seventh embodiment of the wick taken along the length of the wick;

FIG. 16 is a cross-sectional view of an eighth embodiment of the wick taken along the length of the wick;

FIG. 17 is a view of the embodiment of the wick shown in FIG. 15 secured in a retention member mounted in a container for holding a volatile liquid;

FIG. 18 is the wick assembly shown in FIG. 17 showing breakage of the wick in a predetermined location;

FIG. 19 is a side elevational view of a ninth embodiment of the wick of the present invention;

FIG. 19a is an enlarged cross-sectional view taken along line 19a-19a of FIG. 19;

FIG. 19b is an enlarged cross-sectional view taken along line 19b-19b of FIG. 19;

FIG. 20 is an enlarged cross-sectional view of a ninth embodiment of the wick of FIG. 19 secured in a retention member mounted in a container for holding a volatile liquid; and

FIG. 21 is a perspective view of a retention member of the present invention.

### DETAILED DECSRIPTION OF THE INVENTION

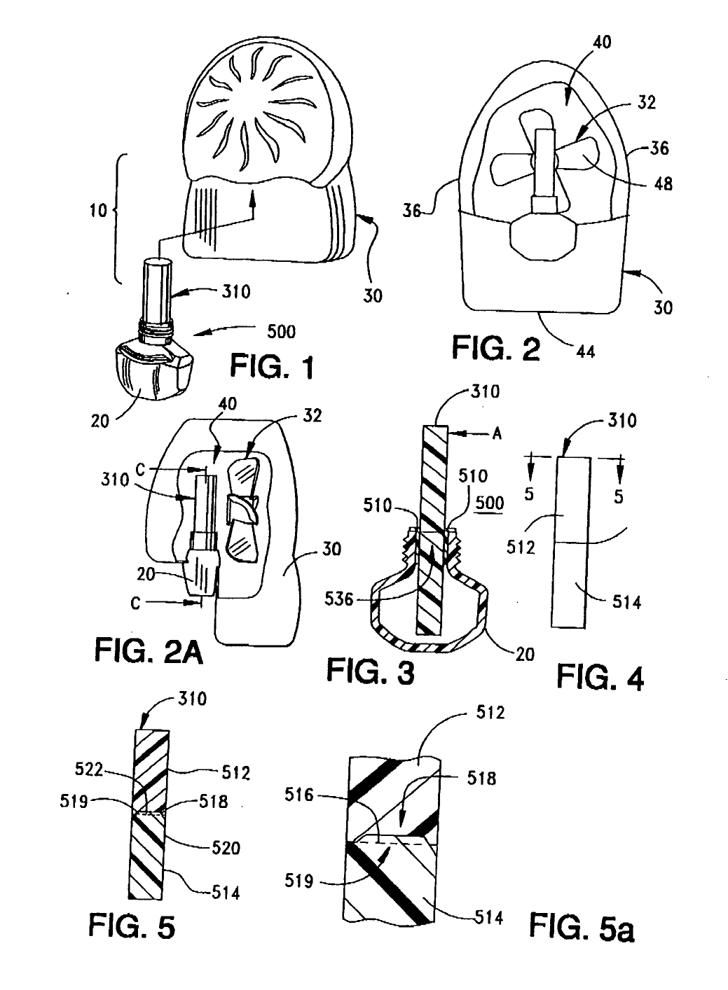

Referring to FIGS. 1, 2 and 2A, a wick assembly, generally designated 500, is designed for use in a volatile liquid dispenser 10. Liquid dispenser 10 is designed to disseminate a volatile liquid, such as a fragrance compound, into a room. The fragrance compound is disseminated via a forced air stream flowing around a wick, generally designated 310, at room ambient temperature. According to the present invention, dispenser 10 includes a housing 30, a motorized fan 32 mounted in housing 30 for generating the air stream, and a wick 310 coupled to housing 30, wherein wick 310 may be formed from a plastic material such as nylon ultra high molecular weight high density polyethylene, or high density polypropylene.

In referring to FIG. 2, housing 30 includes sidewalls 36 and backwall (not shown) to form an interior 40. Base 44 is configured to be able to place dispenser 10 on a flat surface and is a convenient location to position the battery for motorized fan 32. The air stream to impact and pass around wick 310 is generated by the rotation of blades 48.

Portions of housing 30 form an enclosure 40 for fan 32 and for receiving wick 310 of wick assembly 500 into the air stream generated by fan 32. Wick 310 is to be positioned and secured in enclosure 40 formed by housing 30 so as to reside in the air stream generated by fan 32. Wick 310 may be secured in the desired position by coupling wick assembly 500 to dispenser housing 30 using any one of numerous methods. As generally shown in FIG. 2A, container 20 can carry detents or grooves in its sidewalls which will engage housing 30 and thereby releasably secure container 20 to housing 30. Other ways of securing container 20 to housing 30 may include contact adhesives, hook and loop fasteners and the like.

The volatile liquid migrates along wick 310 from the volatile liquid in reservoir 20 to a portion of wick 310 immersed in an air stream generated by fan 32, where it is evaporated from the surface of wick 310 by forced airflow generated by fan 32.

FIGS. 3-20 show various embodiments of wicks and wick assemblies of the present invention. In cross-sectional views showing the wicks alone, the cross section is generally taken along a length of the wick. In showing embodiments of wick assembly 500, the cross-sections will be taken along line C-C shown in FIG. 2A.

In one example, FIG. 3 shows an embodiment of the wick assembly 500 of the present invention which includes wick 310 secured in a container 20 acting as a reservoir for the volatile liquid. Container 20 defines an opening 536 in which wick 310 may be received and secured. When wick assembly 500 includes a container 20, one portion of wick 310 will reside inside container 20 in contact with the volatile liquid held by the container, while another portion of wick 310 resides outside the container. To prevent leakage of volatile liquid from container 20, a seal is formed between wick 310 and container 20 along the portion of wick 20 which transitions from the inside of container 20 to the outside of the container.

Referring to FIGS. 3 and 21, wick 310 may alternatively be secured in a retention member 510 (FIG. 21). Retention member 510 has a generally annular shaped body 651 defining an opening 511 for receiving wick 310 therein, a pair of opposite ends and a flange 650 at one end which abuts an end of container opening 536 (as shown in FIG. 3) when retention member 510 is secured in container opening 536. Retention member 510 may be constructed of a polymeric material, such as an olefin, and other suitable materials. In embodiments of the wick assembly in which wick 310 is secured in retention member 510, seals must be formed between both wick 310 and retention member 510, and retention member 510 and container 20 to prevent leakage of the volatile liquid from container 20.

Forces acting on wick 310 (e.g., during handling of the wick by a user) may be sufficient to cause breakage of the wick. For example, referring to FIG. 3, if the portion of wick 310 residing outside container 20 is subjected to a force acting in a direction indicated by arrow “A” while the portion of wick 310 residing inside container 20 is prevented from moving, the applied force may be sufficient to cause breakage of wick 310. In such a case, it is desirable that the portion of wick 310 residing inside container opening 536 remains secured in opening 536 after breakage of wick 310 in order to prevent leakage of volatile liquid from container 20 through opening 536. To ensure that a portion of wick 310 remains in opening 536 after wick breakage, it is desirable to ensure that wick 310 breaks at a predetermined point along the length of the wick before breakage of any other component of wick assembly 500, such as container 20 or retention member 510, to which wick 310 is secured. The embodiments of wick assembly 500 described below incorporate breakage features therein which facilitate breakage of wick 310 at a predetermined location on the wick when a force is applied to the wick.

In one embodiment, the breakage feature incorporated into wick assemblies described herein comprises a discontinuity formed in an exterior surface of the wick. Referring to FIGS. 4, 5, 5a, 6 and 6a, the discontinuity in wick 310 may be formed by a junction 516 between two adjacent wick segments 512 and 514 secured in abutting contact with each other. Segments 512 and 514 may be constructed of different materials.

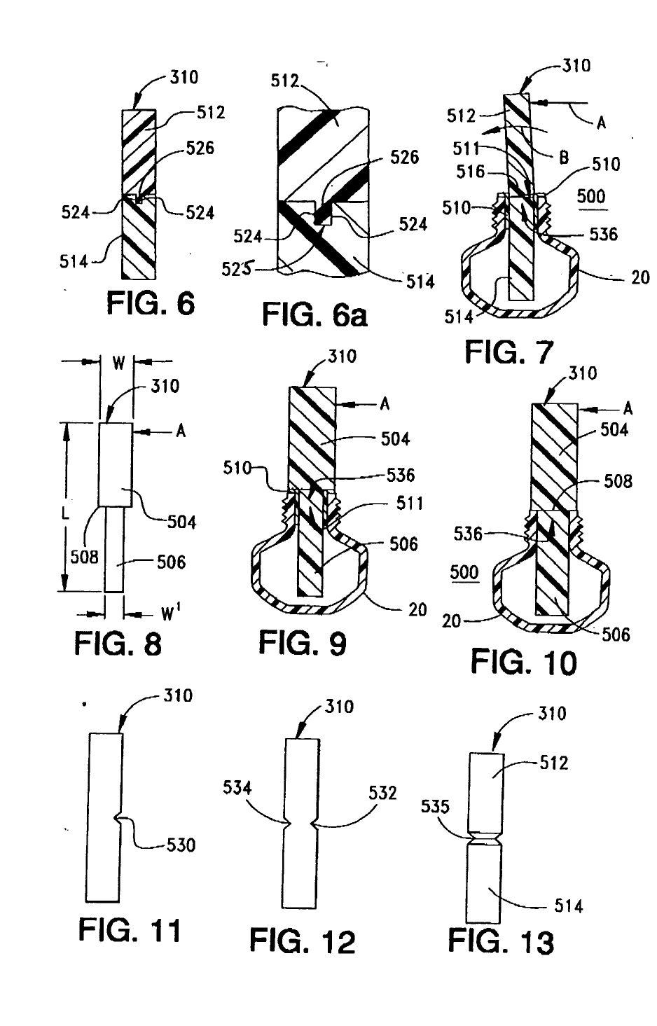

Wick segments 512 and 514 may be secured in abutting contact using any of a variety of methods. However, any method used for securing wick segments 512 and 514 together should ensure a certain minimum, uninterrupted area of contact between wick segments 512 and 514 in order to provide sufficient migration of the volatile liquid from one wick segment to another. For example, as seen in FIGS. 5, 5a, 6 and 6a, segments 512 and 514 may be secured together using an adhesive substance applied at junction 516 between the segments. Referring to FIGS. 5 and 5a, in one example segment 512 has a depression 518 formed therein and segment 512 has an end portion 519 adapted to be received in depression 518. When end portion 519 is received in depression 518, segment 512 is engaged in abutting contact with segment 514 along a surface of segment 512 proximate depression 518. Segments 512 and 514 form a junction 516 on the surface of wick 310. In this embodiment, an adhesive material may be applied along either depression 518 or end portion 519 to bond end portion 519 to depression 518, thereby securing segment 512 in abutting contact with segment 514. In the event an adhesive is used to secure segments 512 and 514 together, an adhesive should be applied intermittently along the respective surfaces to keep open a capillary flow of volatile liquid upwardly through wick 310. Also, the adhesive to be applied should be of lesser resistance to sheer force than the wick 310 material itself.

Referring to FIGS. 6 and 6a, a second embodiment includes a depression 525 in segment 514 has a pair of opposed walls 524. In this embodiment, an end portion of segment 512 includes a tab 526 adapted to be received between opposed walls 524 to form an interference fit therewith, thereby securing segment 512 in abutting contact with segment 514 along their mutual surfaces. Alternatively (or additionally), an adhesive may be applied to opposed walls 524 and/or to tab 526 to aid in securing segments 512 and 514 in abutting contact with each other. Again, the interference fit with or without adhesive should not be as resistant to sheer force as the wick 310 material itself.

FIG. 7 shows an embodiment 500 of the wick assembly of the present invention. In this embodiment, wick 310 of one of the wick embodiments shown in FIGS. 5 and 6 is secured in opening 511 formed in retention member 510. In this embodiment, retention member 510 is secured in an opening 536 defined by container 20. Alternatively, wick 310a may be secured directly in opening 536 of container 20.

For reasons to be explained later, the wick 310 will generally be secured in either retention member 510 or container 20 such that a breakage feature incorporated into the wick resides proximate and end portion of retention member 510 or an end portion of container 20. For example, FIG. 7 shows an embodiment in which wick 310 is positioned in retention member opening 511 such that wick junction 516 resides proximate an end of retention member 510. If wick 310 were to be secured directly in container opening 536, wick 310 would be positioned in container opening 536 such that wick junction 516 would reside proximate an end of container opening 536.

Wick 310 may be secured in retention member opening 511 using an interference fit, adhesive or any one of several other known methods. Any method used to secure wick 310 in retention member opening 511 should aid in preventing leakage of the volatile liquid along a path extending between wick 310 and retention member 510.

Similarly, retention member 510 may be secured in container opening 536 using an interference fit, adhesive or any one of several other known methods. Any method used to secure retention member 510 within container opening 536 should aid in preventing leakage of the volatile liquid along a path extending between retention member 510 and container 20.

FIG. 7 shows the manner in which the breakage features of the present invention operate. The mode of operation shown applies generally to all embodiments of wick 310 and wick assembly 500 described herein.

Referring to FIG. 7, when a force is applied to wick 310 (for example, in the direction indicated by arrow “A”) of wick assembly 500, tensile and compressive stresses are generated in the wick material. Application of a force to wick 310 incorporating one of the breakage features described herein may induce stresses in the wick material near the breakage feature which are greater than the stresses acting on adjacent, relatively homogeneous portions of wick 310. Additionally, the structure of wick 310 in the region of the breakage feature may be less resistant to stresses generated by application of the force due to inclusion of the breakage feature. Thus, formation of a breakage feature in a pre-determined region of wick 310 provides some predictability as to the location of a breaking point of wick 310 when a force is applied to the wick.

As seen in FIG. 7, when one portion 514 of wick is secured within container 20 while a force is applied in the direction of arrow “A” to another portion 512 of wick 310, retention member 510 acts as a pivot about which wick segment 512 may turn in a direction indicated by arrow “B”. Application of sufficient force causes wick segment 512 to disengage from abutting contact with wick segment 514, pivot about retention member 510 and dislodge from retention member opening 511. Keep in mind if a retention member 510 is not employed, container 20 acts as a pivot and wick portion 512 breaks away from container 20 at container 20 opening.

Referring to FIG. 8, in a third embodiment of the wick 310, the discontinuity is formed by a junction 508 between a portion 504 of wick 310 and another, adjacent portion 506 of wick 310 having a different cross sectional area than portion 504. Wick 310 has one or more thickness dimensions W measured in a direction generally transverse to a length dimension L of wick 310. Generally, each thickness dimension will be smaller than length L. As seen in FIG. 8, wick 310 has multiple thickness dimensions W, W′. In a particular version of this embodiment, wick 310 has a cylindrical shape and portion 506 of wick 310 has a diameter different from portion 504 of wick 310.

FIGS. 9 and 10 show the implementation of the third embodiment of wick. In wick assembly 500 (FIG. 9), wick 310 of FIG. 9 is secured in retention member 510, generally an annular plug discussed in more detail below, mounted in container opening 536 of container 20. In wick assembly 500 (FIG. 10), wick 310 is secured directly in container opening 536. Also, a portion 504 of wick 310 residing on a first side of the discontinuity will generally be positioned outside container 20, while another portion of wick 506 residing on an opposite side of the discontinuity will be positioned inside container 20. In this manner, as described above, positioning of the discontinuity or junction 508 in relation to container 20 in this example near the entrance of the opening 536 of container 20, provides predictability as to the location of a breaking point of wick 310 with respect to container 20 when force“A” is applied to wick 310. In this configuration any volatile liquid contained in container 20 will be blocked from spilling out of container 20 upon wick 310 breaking at junction 508.

Referring to FIGS. 9 and 10, when portion 506 of wick is secured within container 20 while a force in a direction indicated by arrow “A” is applied to portion 504 of wick 310, a region of relatively high stresses will be created at junction 508 between adjacent wick portions 504 and 506, due to the relatively abrupt change in cross-sectional area between the adjacent portions. If a sufficient force is applied to wick portion 504, retention member (or container 20) will act as a pivot, about which portion 504 may rotate. Thus, wick 310 will tend to break in the region of junction 508 rather than elsewhere along the length of wick 310.

Referring to FIG. 11, in a fourth embodiment 310 of the wick, the discontinuity is in the form of a notch 530 formed in an exterior surface of wick 310. Notch 530 creates a region of stress concentration in the body of wick 310 during application of a force to wick 310. Thus, the stresses acting on the wick material in the region of this discontinuity due to an applied force will be greater than the stresses acting on adjacent, relatively homogeneous portions of the wick. Consequently, wick 310 will tend to break in the region of notch 530 rather than elsewhere along the length of the wick.

Referring to FIG. 12, in a fifth embodiment of the wick 310, multiple discrete notches 532 and 534 may be formed in the exterior surface of the wick. Although more notches may be formed in wick 310, only two notches are shown in FIG. 12 for purposes of clarity. In a sixth embodiment of the wick 310, shown in FIG. 13, a continuous notch 535 may be formed extending about a circumference of the body of wick 310.

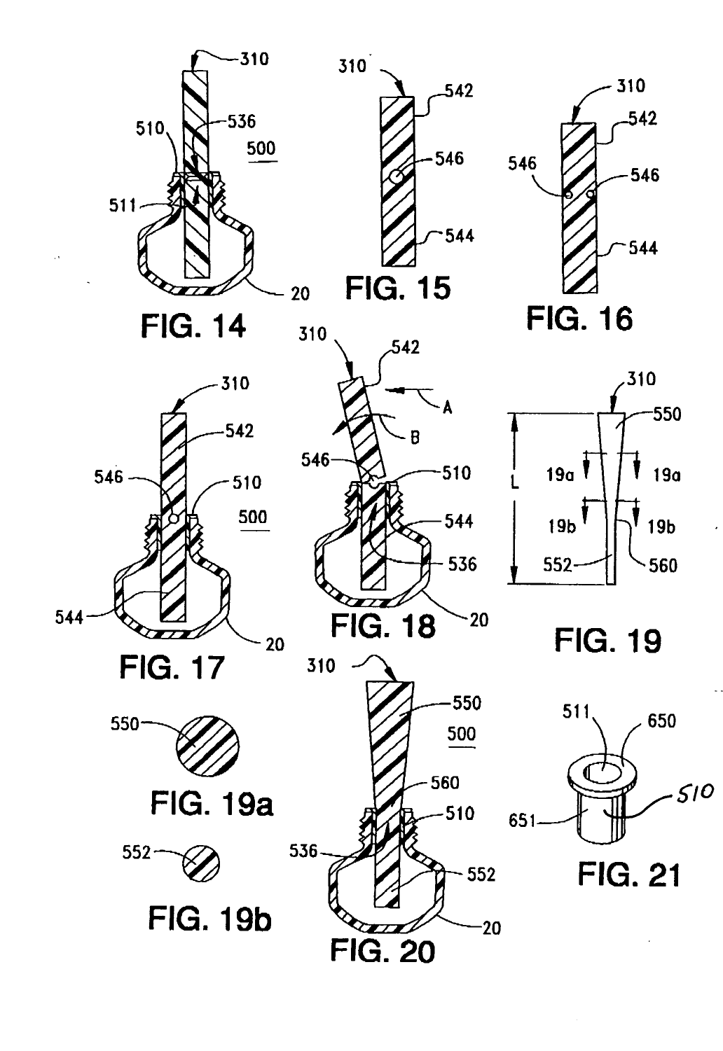

FIG. 14 shows employment of FIG. 13 wick 310 mounted in retention member 510 which is secured, in turn, in container opening 536 as described above. Wick 310 may alternatively be secured directly in container opening 511. Referring to FIG. 14, wick 310 is secured in retention member opening 511 such that notch 535 resides proximate an end of either retention member 510 or container opening 536 to provide a predictable pivot and breakage location along the wick, as described above.

In another embodiment, the breakage feature incorporated into wick assemblies described herein comprises an absence of material formed in an interior portion of the wick. Referring to FIGS. 15 and 16 of the seventh and eighth embodiments, respectively, wicks 310 have elongated bodies and opposite ends 542 and 544. As stated above, wicks 310 may be formed from a plastic material such as nylon or high density polyethylene or the like.

In these embodiments, one or more absences 546 of the wick material are selectively positioned within the wick body intermediate ends 542 and 544. When the wick is formed from a plastic material, for example, absence (or absences) 546 may be formed by a blow molding process. In this process, a blow pin is inserted into a molten wick secured in a mold. Compressed air is then injected into the interior of the wick to displace a portion of the plastic in the wick interior.

Absence 546 creates a portion of the wick having a cross-sectional area less than adjacent portions of the wick which are substantially filled with the material from which the wick is formed. The pressure acting on the wick material along this reduced cross-sectional area due to an applied force will be greater than the pressure acting on adjacent, relatively homogeneous portions of the wick. Consequently, this reduced cross-sectional area provides less resistance to breakage than adjacent portions of the wick. In this manner, the forming of absence 546 in a pre-determined region of the wick provides some predictability as to the location of a breaking point of the wick when a force is applied to the wick.

FIG. 18 shows employment of the embodiment of wick 310 shown in FIG. 15. Wick 310 is secured in a retention member 510. Wick 310 may alternatively be secured directly in an opening 536 of a container 20 for holding the volatile liquid. As seen in FIG. 18, wick segment 542 resides outside container 20 while wick segment 544 is secured within container 20. In general, wick is positioned with respect to an end portion of retention member 510 or container 20 such that an exterior surface of wick 310 which is proximate interior absence 546 resides proximate the end portion of retention member 510 or container 20.

As seen in FIG. 18, when a sufficient force is applied to the exposed end 542 of wick 310 in the direction indicated by arrow “A”, retention member 510 acts as a pivot about which wick segment 542 will tend to rotate, in a direction indicated by arrow “B”. Thus, when a sufficient force is applied to wick 310, the wick will tend to break proximate retention member 510, in the manner described previously.

Referring to FIGS. 19, 19a, 19b and 20, a ninth embodiment of the wick 310 comprises two segments 550 and 552 and a length L along which successive adjacent cross sectional areas of wick 310 taken generally transverse to length L vary along the length of the wick. As seen in the embodiments described above, wick 310 may be secured in either a retention member 510 or in a container 20. When wick 310 is secured in a retention member 510 or container 20, the variation in cross-sectional area of wick 310 generally occurs between segment 550 of wick 310 residing outside container 20, and a portion 560 residing proximate an end portion of either retention member 510 or container 20.

Referring to an example of this embodiment of wick 310 successive adjacent cross sectional areas of wick 310 vary uniformly along the length L of wick 310 between segment 550 of wick 310 and position 560 on the wick. In this embodiment, the areas of successive cross sections of wick 310 taken transverse to the wick will vary at a uniform rate, generally depending on the convergence or divergence of opposing exterior surfaces of the wick. In a particular embodiment, perimeters of successive adjacent cross sections of the wick taken generally transverse to the length will have substantially the same shape. For example, perimeters of successive adjacent cross sections taken along lines 19a-19a and 19b-19b (FIG. 19) will have substantially similar shapes, as shown in FIGS. 19a and 19b.

Referring to FIG. 20, wick 310 may have a pre-determined location 560 along its length at which a cross sectional area of the wick taken generally transverse to the length is less than or equal to a cross sectional area of wick 310 taken generally transverse to length L of wick 310 at any other location along the length of the wick. This location defines a cross section 560 having smallest area of any cross section taken transverse to length L of wick 310 in comparison to the wick portion 550 positioned outside of container 20. Due to the relatively small amount of wick material resisting a force applied to wick 310, the wick will tend to break at location 560 having the smallest cross sectional area, of the portion of the wick 550 outside of container 20, when a force is applied to wick segment 550.

As seen in FIG. 20, when wick 310 is secured in either retention member 510 or container 20 as described above, location 560 on wick 310 may be positioned proximate an end portion of either retention member 510 or container 20 to provide a predictable pivot and breakage location along length L of wick 310.

In the above-described embodiments, the wick is longitudinal, has a longitudinal axis and a curved exterior surface. In alternative embodiments, the wick may be formed such that a cross-section of the wick taken perpendicular to the longitudinal axis has any one of a variety of simple geometric shapes, such as a circle, a diamond, a rectangle, a hexagon, an octagon, an oval or a triangle.

Industrial Applicability

The present invention provides various features incorporated into a wick assembly which facilitate the breakage of a porous wick at a predetermined location on the wick when a force is applied to the wick. Generally, the wick is secured within an opening of a container for holding a volatile liquid, with a portion of the wick residing inside the container and a portion of the wick residing outside the container. The features described herein facilitate breakage of the wick before breakage of any other portion of the wick assembly (for example, the container). These features also help ensure that the portion of the wick secured in the container opening remains secured in the opening after wick breakage, thereby preventing leakage of the volatile liquid through the container opening.

It should be understood that the preceding is merely a detailed description of various embodiments of this invention and that numerous changes to the disclosed embodiment can be made in accordance with the disclosure herein without departing from the spirit or scope of the invention. The preceding description, therefore, is not meant to limit the scope of the invention. Rather, the scope of the invention is to be determined only by the appended claims and their equivalents.

1. A wick assembly for use in a dispenser for a volatile liquid, comprising:
a wick having an external surface; and
a discontinuity formed in the surface providing a location in the wick of less resistance to a force applied to the wick than a location adjacent to the discontinuity.

2. The wick assembly of claim 1 wherein the wick is elongate.

3. The wick assembly of claim 2 wherein the wick has a thickness measured in a direction generally transverse to a length of the wick and is of a lesser dimension than the length.

4. The wick assembly of claim 3 wherein the location and the adjacent location have different cross sectional areas.

5. The wick assembly of claim 4 wherein the wick has a cylindrical shape in which the location has a smaller diameter than the adjacent location.

6. The wick assembly of claim 1 wherein a perimeter of a cross section of the wick is a simple geometric shape.

7. The wick assembly of claim 6 wherein the geometric shape includes one of a circle, a diamond, a rectangle, a hexagon, an octagon, an oval and a triangle.

8. The wick assembly of claim 1 in which the discontinuity includes a junction between two portions of the wick.

9. The wick assembly of claim 1 wherein the discontinuity includes a notch.

10. The wick assembly of claim 1 wherein the wick operates at ambient room temperature.

11. The wick assembly of claim 9 wherein the notch is positioned about a circumference of the wick.

12. The wick assembly of claim 1 wherein at least a portion of the wick is constructed of high density polyethylene.

13. The wick assembly of claim 1 further comprising a retention member defining an opening adapted to be received in another opening of a container for holding the volatile liquid and in which the opening of the retention member receives a portion of the wick therein.

14. The wick assembly of claim 13 wherein the portion of the wick received in the opening of the retention member forms an interference fit with the retention member.

15. The wick assembly of claim 13 wherein the discontinuity in the surface of the wick is positioned proximate the retention member.

16. The wick assembly of claim 13 wherein the discontinuity is positioned proximate to the other opening of the container.

17. The wick assembly of claim 1 comprising a wick having a segment and another segment secured in abutting contact with the segment, and wherein the discontinuity is formed by a junction between the segments.

18. The wick assembly of claim 17 wherein the two abutting segments are secured together using an adhesive.

19. The wick assembly of claim 17 wherein the segment has a depression formed in an end portion and the other segment has another end portion configured to be compatibly received in the depression, and wherein the other segment is engaged in abutting contact with the segment along a surface of the segment proximate the depression.

20. The wick assembly of claim 19 further comprising an adhesive material residing in the junction between the segment and the other segment.

21. The wick assembly of claim 19 wherein the depression has a pair of opposed walls and the other segment end portion is received between the opposed walls to form an interference fit therewith, thereby securing the other segment in abutting contact with the segment.

22. The wick assembly of claim 17 further including a retention member defining an opening adapted to receive a portion of the segment and a portion of the other segment therein to secure the portion of the other segment in abutting contact with the portion of the segment.

23. The wick assembly of claim 22 wherein the retention member has a pair of opposed ends, and the portion of the other segment is secured in abutting contact with the portion of the segment such that a junction between the segments resides proximate one of the retention member ends.

24. The wick assembly of claim 17 further including a container for holding the volatile liquid, the container defining an opening adapted to receive a portion of the segment and a portion of the other segment therein to secure the portion of the other segment in abutting contact with the portion of the segment.

25. The wick assembly of claim 24 wherein the portion of the other segment is secured in abutting contact with the portion of the segment such that a junction between the segments resides proximate the opening defined by the container.

26. A wick assembly for use in a dispenser for a volatile liquid, comprising:
a wick having a body constructed of a material; and
an absence of the material selectively positioned at a location within the body.

27. The wick assembly of claim 26 wherein the wick is elongated and has a pair of opposite ends, and the absence is formed intermediate the opposite ends of the wick

28. The wick assembly of claim 26 wherein the wick is formed from high density polyethylene.

29. The wick assembly of claim 26 wherein the wick is formed from nylon.

30. The wick assembly of claim 26 wherein the wick is formed from high density polypropylene.

31. The wick assembly of claim 26 wherein a cross section of the wick taken generally transverse to a length of the wick has a simple geometrical shape.

32. The wick assembly of claim 31 wherein the shape is one of a circle, a diamond, a rectangle, a hexagon, an octagon, an oval and a triangle.

33. The wick assembly of claim 26 further comprising a retention member defining an opening in which the retention member is adapted to be received in another opening of a container for holding the volatile liquid in which the opening of the retention member receives a portion of the wick therein.

34. The wick assembly of claim 33 wherein the portion of the wick received in the opening of the retention member forms an interference fit with the retention member.

35. The wick assembly of claim 33 wherein the portion of the wick received in the opening of the retention member resides proximate the absence of the wick material.

36. The wick assembly of claim 33 wherein the absence of the material is positioned proximate the other opening of the container.

37. The wick assembly of claim 36 wherein the retention member forms interference fit with the container.

38. The wick assembly of claim 26 further comprising a container for holding the volatile liquid, the container defining another opening adapted to receive a portion of the wick therein.

39. The wick assembly of claim 26 wherein the wick assembly operates at ambient room temperature.

40. A wick assembly for use in a dispenser for a volatile liquid, comprising:
a wick having opposing ends and a length therebetween in which successive adjacent cross sectional areas of the wick taken generally transverse to the length, vary in cross section area along said length of the wick between one end of the wick and a position on the wick residing proximate an opening of a container for holding the volatile liquid.

41. The wick assembly of claim 40 wherein successive adjacent cross sectional areas of the wick vary uniformly along the length of the wick between the one end of the wick and the position on the wick.

42. The wick assembly of claim 40 wherein the one end of the wick resides outside the container.

43. The wick assembly of claim 40 wherein the wick is formed from high density polyethylene.

44. The wick assembly of claim 40 wherein the wick is formed from nylon.

45. The wick assembly of claim 40 wherein the wick is formed from high density polypropylene.

46. The wick assembly of claim 40 wherein a cross section of the wick taken generally transverse to the length of the wick has a simple geometrical shape.

47. The wick assembly of claim 46 wherein the shape is one of a circle, a diamond, a rectangle, a hexagon, an octagon, an oval and a triangle.

48. The wick assembly of claim 40 wherein perimeters of successive adjacent cross sections of the wick taken generally transverse to the length have substantially the same shape.

49. The wick assembly of claim 40 further comprising a retention member defining an opening adapted to be received in another opening of a container for holding the volatile liquid and in which the opening of the retention member receives a portion of the wick therein.

50. The wick assembly of claim 49 wherein the wick has a pre-determined location along the length of the wick at which a cross sectional area of the wick taken generally transverse to the length of the wick is less than or equal to a cross sectional area of the wick taken generally transverse to the length of the wick at any other location along the length of the wick.

51. The wick assembly of claim 50 wherein the pre-determined location on the wick is positioned proximate the retention member.

52. The wick assembly of claim 50 further comprising a container for holding the volatile liquid, the container defining another opening adapted to receive a portion of the retention member therein.

53. The wick assembly of claim 52 wherein the portion of the retention member forms an interference fit with the container.

54. The wick assembly of claim 50 further comprising a container for holding the volatile liquid, the container defining another opening adapted to receive a portion of the wick therein.

55. The wick assembly of claim 54 wherein the portion of the wick forms an interference fit with the container.

56. The wick assembly of claim 55 wherein the pre-determined location of the wick is positioned proximate the container opening.

57. A wick assembly for use in a dispenser for volatile liquid, comprising:
a wick adapted to be positioned to extend from an inside of a container, for holding the volatile liquid, through an opening defined in the container and to an outside of the container; and
means for enabling the wick to separate into at least two portions wherein one of the portions blocks the opening of the container.

58. The wick assembly of claim 57 wherein the means for enabling the wick to separate comprises an absence of the material selectively positioned within the body.

59. The wick assembly of claim 57 wherein the means for enabling the wick to separate comprises a notch formed in an exterior surface of the wick

60. The wick assembly of claim 57 wherein the means for enabling the wick to separate comprises a seam formed between a first and a second portions of the wick in which the second portion of the wick abuts the first portion.

61. The wick assembly of claim 57 wherein the wick has a first cross section generally transverse to a length of the wick, the first cross section having a first cross sectional area, and the means for enabling the wick to separate comprises providing a second cross section of the wick generally transverse to a length of the wick and adjacent the first cross section in which the second cross section has a second cross sectional area less than the first cross sectional area.
